(19) United States
(12) Patent Application Publication
(10) Pub. No.: US 2023/0167166 A1
(43) Pub. Date: Jun. 1, 2023

(54) TARGETED THERAPY FOR THE TREATMENT & PREVENTION OF LIFE-THREATENING COMPLICATIONS OF INFECTION

(71) Applicant: MEMORIAL SLOAN KETTERING CANCER CENTER, New York, NY (US)

(72) Inventors: Larry Norton, New York, NY (US); Ross L Levine, New York, NY (US); Kelly L Bolton, St. Louis, MO (US); Ahmet Zehir, New York, NY (US)

(21) Appl. No.: 17/920,902

(22) PCT Filed: Apr. 26, 2021
(86) PCT No.: PCT/US21/29097

§ 371 Date: Oct. 24, 2022

Related U.S. Application Data
(60) Provisional application No. 63/015,297, filed on Apr. 24, 2020.
(60) Provisional application No. 63/070,199, filed on Aug. 25, 2020.

(51) Int. Cl. C07K 16/10; A61P 31/14; A61P 11/00; A61P 29/00; C07K 16/28; C07K 16/24; A61K 38/20; A61K 38/17; A61K 31/519; A61K 31/529; C12Q 1/6883; C12Q 1/70

(52) U.S. Cl. CPC C07K 16/10; A61P 31/14; A61P 11/00; A61P 29/00; C07K 16/2866; C07K 16/248; A61K 38/2006; C07K 16/245; A61K 38/1793; C07K 16/2893; A61K 31/519; C07K 16/241; A61K 31/529; C07K 16/24; C12Q 1/6883; C12Q 1/70; C07K 2317/24; A61K 39/00

(57) ABSTRACT

The present invention provides a variety of methods for the identification and/or treatment of subjects that are at risk for developing life-threatening complications of SARS-CoV-2 infection and other infections. Such methods involve determining if a subject has clonal hematopoiesis.

### CROSS-REFERENCE TO RELATED APPLICATIONS

This application claims the benefit of priority of U.S. Provisional Patent Application No. 63/015,297 filed on Apr. 24, 2020 and U.S. Provisional Patent Application No. 63/070,199 filed on Aug. 25, 2020, the content of each of which is hereby incorporated by reference in its entirety.

### STATEMENT REGARDING FEDERALLY SPONSORED RESEARCH

This invention was made with government support under CA241318 and CA008748 awarded by the National Institutes of Health. The government has certain rights in the invention.

### INCORPORATION BY REFERENCE

For the purposes of only those jurisdictions that permit incorporation by reference, all of the references cited in this disclosure are hereby incorporated by reference in their entireties. In addition, any manufacturers' instructions or catalogues for any products or active agents cited or mentioned herein are incorporated by reference. Documents incorporated by reference into this text, or any teachings therein, can be used in the practice of the present invention.

### BACKGROUND

The disease COVID-19—which is caused by the SARS-CoV-2 coronavirus—was declared to have reached pandemic status by the World Health Organization (WHO) in March 2020. Mortality from COVID-19 is currently estimated to be around 2%, although the mortality rate depends on numerous factors. Cytokine release syndrome (CRS), a massive outpouring of cytokines including IL-6, is one of the main causes of the most common lethal aspect of COVID-19—i.e., inflammatory damage of organs including profound lung inflammation resulting in the oxygenation defect called acute respiratory distress syndrome (ARDS). The anti-IL-6 drug tocilizumab is in clinical trials for COVID-19, but so far its use has been reserved for severe cases³². Similarly, while monoclonal antibodies have been granted emergency use authorization in the U.S. for treatment of COVID-19, so far their use has been limited to severe cases. The institution of such therapies late in the evolution of the disease, when much organ damage has already occurred, is not ideal. It would be advantageous to start COVID-19 therapy before severe respiratory distress ensues—even in young patients without known risk factors for severe COVID-19. However, to do so would require that high-risk cases be identified earlier in the course of the disease than is currently possible. There are also numerous other infections (including bacterial, viral and fungal infections) that can also lead to severe and life-threatening complications similar to those occurring in COVID-19 patients—including sepsis, CRS and ARDS—and for which it would be advantageous to commence therapy before such severe symptoms develop. Accordingly, there is an urgent need in the art for new therapeutic options for the prevention and/or early treatment of life-threatening complications of severe infections—both in patients with COVID-19 and in patients with other infections. The present invention addresses this need.

### SUMMARY OF THE INVENTION

Some of the main embodiments of the present invention are summarized below. Additional embodiments are described in the Detailed Description, Examples, Figures, Brief Description of the Figures and Claims sections of this disclosure. The description in each section of this patent disclosure, regardless of any heading or sub-heading titles, is intended to be read in conjunction with all other sections. Furthermore, the various embodiments described in each section of this disclosure can be combined in various different ways, and all such combinations are intended to fall within the scope of the present invention. Any sub-headings provided throughout this patent disclosure are not intended to denote limitations of the various aspects or embodiments of the invention, which are to be understood by reference to the specification as-a-whole.

It is now well established that people without overt hematological malignancies sometimes demonstrate clonal hematopoiesis (CH), also called clonal hematopoiesis of indeterminate potential (CHIP)—a condition in which circulating mutant white blood cells clonally expand in an individual and predispose that individual to the subsequent development of hematological malignancies and/or atherosclerotic cardiovascular disease. The present invention is based, in part, on the novel hypothesis that CH may be associated with severe COVID-19 including the life-threatening complications of SARS-CoV-2-infection: ARDS and CRS, and that the presence of CH can be used for the identification of those patients that are at risk of developing severe COVID-19 such that treatment can be initiated early—even if the absence of, or prior to the onset of, ARDS, CRS or other symptoms of severe COVID-19. The data presented in the Examples section of this patent disclosure confirms the relationship between CH and the risk of severe COVID-19 and other infections in a human clinical study. Building on these hypotheses and data, the present invention provides a variety of methods for the identification and treatment of subjects that are at risk for developing life-threatening complications of SARS-CoV-2-infection and other infections.

Accordingly, in one embodiment, the present invention provides a method of treating COVID-19 in a subject in need thereof, the method comprising: administering an effective amount of an anti-cytokine agent to a subject with COVID-19, wherein the subject has clonal hematopoiesis (CH), thereby treating COVID-19 in the subject. In some such embodiments such methods further comprise performing an assay to determine if the subject has CH prior to administering the anti-cytokine agent to the subject. Similarly, in some such embodiments such methods further comprise performing an assay to determine if the subject is infected with a SARS-CoV-2 virus.

In another embodiment, the present invention provides a method of treating COVID-19 in a subject in need thereof, the method comprising: administering an effective amount of an anti-COVID-19 antibody therapy to a subject with COVID-19, wherein the subject has clonal hematopoiesis (CH), thereby treating COVID-19 in the subject. In some such embodiments such methods further comprise performing an assay to determine if the subject has CH prior to administering the anti-COVID-19 antibody therapy to the subject. Similarly, in some such embodiments such methods further comprise performing an assay to determine if the subject is infected with a SARS-CoV-2 virus.

In another embodiment, the present invention provides a method of treating or preventing CRS, ARDS or sepsis associated with an infection in a subject in need thereof, the method comprising: administering an effective amount of an anti-cytokine agent to a subject with an infectious disease, wherein the subject has clonal hematopoiesis (CH), thereby treating CRS, ARDS or sepsis associated with an infection in the subject. In some such embodiments such methods further comprise performing an assay to determine if the subject has CH prior to administering the anti-cytokine agent to the subject. Similarly, in some such embodiments such methods further comprising performing an assay to determine if the subject is infected with a SARS-CoV-2 virus.

In yet another embodiment, the present invention provides a method of determining if a subject infected with a SARS-CoV-2 virus is a candidate for initiation of anti-cytokine therapy, the method comprising: determining if the subject has CH, wherein, if the subject has CH the subject is a candidate for initiation of anti-cytokine therapy.

In a further embodiment, the present invention provides a method of determining if a subject infected with a SARS-CoV-2 virus is a candidate for initiation of anti-COVID-19 antibody therapy, the method comprising: determining if the subject has CH, wherein, if the subject has CH the subject is a candidate for initiation of anti-COVID-19 antibody therapy.

In yet another embodiment, the present invention provides a method of determining if a subject with an infection is a candidate for initiation of anti-cytokine therapy, the method comprising: determining if the subject has CH, wherein, if the subject has CH the subject is a candidate for initiation of anti-cytokine therapy.

In those embodiments of the present invention that involve determining if a subject has CH, or performing an assay to determine if the subject has CH (i.e., a “CH assay”), any suitable method or assay for determining if a subject has CH can be used. In some embodiments the CH assay detects clonally expanded hematopoietic cells having a variant allele/acquired mutation. In some embodiments the CH assay comprises performing a molecular analysis and/or DNA sequencing. In some embodiments the CH assay comprises determining the sequence of DNA of circulating leukocytes from a subject from a subject. In some embodiments the CH assay comprises determining the sequence of cell-free DNA (e.g., in a blood s ample) from a subject. In some embodiments such molecular analysis and/or DNA sequencing is performed to determine the frequency of variant alleles. In some embodiments the variant allele can be any variant allele/acquired mutation that associated with CH, for example any of those alleles described in the Examples section of this disclosure or known in the art. (CH-associated variant alleles include, but are not limited to, those described in Bolton et al., “Cancer therapy shapes the fitness landscape of clonal hematopoiesis.” Nat Genet. 2020, Vol. 52(11):1219-1226, the contents of which are hereby incorporated by reference). In some embodiments the CH assay comprises determining the sequence of, and/or determining the frequency of variant alleles in, a leukemia-associated gene. In some embodiments the CH assay comprises determining the sequence of, and/or determining the frequency of variant alleles in, the DNMT3A, TET2, or ASXL1 gene. In some embodiments the CH assay comprises determining the sequence of, and/or determining the frequency of variant alleles in, the TET2 gene. In some embodiments the CH assay is performed in a subject that is not exhibiting symptoms of ARDS or CRS—i.e., in the absence of ARDS or CRS. In some embodiments the CH assay is performed prior to the onset of, symptoms of ARDS or CRS in the subject.

In those embodiments of the present invention that involve determining if a subject has CH, the subject is typically determined to have CH if the subject has a variant allele frequency (VAF) of ≥about 2 percent. In some embodiments the subject is determined to have CH if the subject has an elevated variant allele frequency (VAF). In some embodiments the subject is determined to have CH if the subject has a variant allele frequency (VAF) of ≥about 0.5 percent. In some embodiments the subject is determined to have CH if the subject has a variant allele frequency (VAF) of ≥about 1.0 percent. In some embodiments the subject is determined to have CH if the subject has a variant allele frequency (VAF) of ≥about 1.5 percent. In some embodiments the subject is determined to have CH if the subject has a variant allele frequency (VAF) of ≥about 2 percent. In some embodiments the subject is determined to have CH if the subject has a variant allele frequency (VAF) of ≥about 3 percent. In some embodiments the subject is determined to have CH if the subject has a variant allele frequency (VAF) of ≥about 4 percent. In some embodiments the subject is determined to have CH if the subject has a variant allele frequency (VAF) of ≥about 5 percent. In some embodiments the variant allele can be any variant allele/acquired mutation that is known to be associated with CH, for example any of those alleles described in the Examples section of this disclosure or known in the art. In some embodiments the subject is determined to have CH if the subject has a VAF of ≥about 0.5 percent, or ≥about 1.0 percent, or ≥about 1.5 percent, or ≥about 2.0 percent, or ≥about 3 percent, or ≥about 4 percent, or ≥about 5 percent, of an of an acquired mutation of a leukemia-associated gene. In some embodiments the subject is determined to have CH if the subject has a VAF of ≥about 0.5 percent, or ≥about 1.0 percent, or ≥about 1.5 percent, or ≥about 2.0 percent, or ≥about 3 percent, or ≥about 4 percent, or ≥about 5 percent, of an acquired mutation of the DNMT3A, TET2, and/or ASXL1 genes. In some embodiments the subject is determined to have CH if the subject has a VAF of ≥about 0.5 percent, or ≥about 1.0 percent, or ≥about 1.5 percent, or ≥about 2.0 percent, or ≥about 3 percent, or ≥about 4 percent, or ≥about 5 percent, of an of an acquired mutation of the TET2 gene. In some embodiments the variant allele/acquired mutation is present in circulating leukocytes. In some embodiments the variant allele/acquired mutation is present in cell free DNA (e.g., as present in the blood or in a blood sample). Methods of determining variant allele frequencies are known in the art, for example as described in Zehir A, et al., “Mutational landscape of metastatic cancer revealed from prospective clinical sequencing of 10,000 patients,” Nat. Med. 2017, vol. 23(6), pp. 703-713, the contents of which are hereby incorporated by reference.

In those embodiments of the present invention that involve performing an assay to determine if the subject is infected with a SARS-CoV-2 virus (i.e., a “SARS-CoV-2 assay”), any suitable assay for detection of SARS-CoV-2 infection can be used. In some embodiments the SARS-CoV-2 assay comprises performing viral culture to detect the SARS-CoV-2 virus. In some embodiments the SARS-CoV-2 assay comprises detecting nucleic acids of the SARS-CoV-2 virus. In some embodiments the SARS-CoV-2 assay comprises detecting nucleic acids of the SARS-CoV-2 virus by PCR. In some embodiments the SARS-CoV-2 assay comprises detecting an antigen or antigens of the SARS-CoV-2 virus. In some embodiments the SARS-CoV-2 assay comprises detecting antibodies against the SARS-CoV-2 virus. Numerous suitable tests are known in the art, including multiple that have been approved or granted emergency use authorization by the U.S. Food and Drug Administration (i.e., the FDA) and/or other national or international regulatory agencies.

In those embodiments of the present invention that involve anti-cytokine agents, the anti-cytokine agent can be any suitable anti-cytokine agent. Numerous suitable agents are known in the art, including multiple that have been approved or granted emergency use authorization by the U.S. Food and Drug Administration (i.e., the FDA) and/or other national or international regulatory agencies. In some embodiments the anti-cytokine agent is an IL-6 inhibitor. In some embodiments the anti-cytokine agent is selected from the group consisting of tocilizumab, siltuximab, anakinra, canakinumab, rilonacept, rituximab, alemtuzumab, ruxolitinib, fedratinib, pacritinib, tofacitinib, tadekinig-alpha, emapalumab, infliximab, etanercept, ronatinib, and corticosteroids.

In those embodiments of the present invention that involve administration of an effective amount of an anti-cytokine agent to a subject, the anti-cytokine agent can be administered at any suitable time—for example as determined by a medical professional. In some embodiments the anti-cytokine agent is administered to the subject as early as possible in the course of the infection and/or as soon as possible after the subject has been determined to have CH. In some embodiments the anti-cytokine agent is administered to the subject in the absence of symptoms of severe COVID-19 or acute respiratory distress syndrome (ARDS) or cytokine release syndrome (CRS). In some embodiments the anti-cytokine agent is administered to a subject in the absence of symptoms of severe COVID-19 or acute respiratory distress syndrome (ARDS) or cytokine release syndrome (CRS).

In those embodiments of the present invention that involve anti-COVID-19 antibodies or anti-COVID-19 antibody therapy, the anti-COVID-19 antibodies can be any suitable anti-COVID-19 antibodies or a cocktail of such antibodies. Several such antibodies are known in the art, including several that have been approved or granted emergency use authorization by the U.S. Food and Drug Administration (i.e., the FDA) and/or other national or international regulatory agencies, for the treatment of COVID-19—including bamlanivimab, etesevimab, casirivimab and imdevimab. Accordingly, in some embodiments the anti-COVID-19 antibody or anti-COVID-19 antibody therapy comprises one or more of bamlanivimab, etesevimab, casirivimab and imdevimab. In some embodiments the anti-COVID-19 antibody or anti-COVID-19 antibody therapy comprises the combination of casirivimab and imdevimab. In some embodiments the anti-COVID-19 antibody or anti-COVID-19 antibody therapy comprises the combination of bamlanivimab and etesevimab.

In some embodiments the anti-COVID-19 antibodies are or comprise neutralizing antibodies against the spike protein of the SARS-CoV-2 virus. Several such antibodies are known in the art, including some that have been approved or granted emergency use authorization by the U.S. Food and Drug Administration (i.e., the FDA) and/or other national or international regulatory agencies for the treatment of COVID-19, including bamlanivimab, etesevimab, casirivimab and imdevimab.

In those embodiments of the present invention that involve administration of an effective amount of anti-COVID-19 antibodies or anti-COVID-19 antibody therapy to a subject, the anti-COVID-19 antibodies can be administered at any suitable time—for example as determined by a medical professional. In some embodiments the anti-COVID-19 antibodies are administered to the subject as early as possible in the course of the infection and/or as soon as possible after the subject has been determined to have CH. In some embodiments the anti-COVID-19 antibodies are administered to the subject in the absence of symptoms of severe COVID-19 or acute respiratory distress syndrome (ARDS) or cytokine release syndrome (CRS). In some embodiments the anti-COVID-19 antibodies are administered to a subject in the absence of symptoms of severe COVID-19 or acute respiratory distress syndrome (ARDS) or cytokine release syndrome (CRS).

In those embodiments of the present invention that involve treatment of infection, or determining if a subject with an infection is a candidate for initiation of therapy, in some embodiments the infection is any infection that results in, or has the potential to result in, ARDS, CRS or sepsis. In some embodiments the infection is a viral infection. In some embodiments the infection is a bacterial infection. In some embodiments the infection is a fungal infection. In some embodiments the infection is a SARS-CoV-2 infection. In some embodiments the infection is a Clostridium Difficile infection. In some embodiments the infection is a Streptococcus infection. In some embodiments the infection is an Enterococcus infection.

These and other embodiments of the present invention are further described in the Detailed Description, Examples, Figures, Brief Description of the Figures and Claims sections of this disclosure.

### BRIEF DESCRIPTION OF THE FIGURES

FIG. 1. Association between CH and COVID-19 severity. Shown are the results from logistic regression adjusted for age, gender, race, smoking, diabetes, cardiovascular disease, COPD/asthma, cancer primary site (if history of malignancy), exposure to cytotoxic cancer therapy for the MSK and KoCH cohorts. Summary statistics for a fixed effects meta-analysis are shown.

FIG. 2A-B. Association between CH and risk of infection in solid tumor patients. FIG. 2A—Volcanoplot of the log(Hazard ratio) of infection with CH using multivariable cox proportional hazards regression. FIG. 2B—Association between CH subtype defined by putative driver status and risk of Clostridium Difficile and Streptococcus/Enterococcus infection using cox proportional hazards regression. All models were adjusted for age, gender, race, smoking, diabetes, cardiovascular disease, COPD/asthma, cancer primary site (if history of malignancy), exposure to cytotoxic cancer therapy.

FIG. 3. Association between CH and COVID-19 severity. Shown are the results from logistic regression adjusted for age, gender, race, smoking, diabetes, cardiovascular disease, COPD/asthma, cancer primary site (if history of malignancy), exposure to cytotoxic cancer therapy for the MSK and Korea Consortia. Summary statistics for a fixed effects meta-analysis are shown.

FIG. 4-B. Number of mutations and variant allele fraction of CH by COVID-19 Status. FIG. 4A—Number of CH mutations among those with severe and non-severe Covid-19. FIG. 4B—VAF of CH mutations by COVID-19 severity and infection status.

FIG. 5. Association between CH and COVID-19 severity stratified by the number of mutations. Shown are the results from logistic regression comparing the odds ratios of severe COVID-19 among those with one mutation and those with two or more mutations. Models were adjusted for age, gender, race, smoking, diabetes, cardiovascular disease, COPD/asthma, cancer primary site (if history of malignancy), exposure to cytotoxic cancer therapy for the MSK and Korea Consortia. Summary statistics for a fixed effects meta-analysis are shown.

FIG. 6. Association between maximum VAF of CH-mutation(s) and COVID-19 severity. Shown are the results from logistic regression comparing the odds ratios of severe Covid-19 among those with one or more CH mutations<5% VAF compared to no CH and CH with a VAF>5% and no CH. Models were adjusted for age, gender, race, smoking, diabetes, cardiovascular disease, COPD/asthma, cancer primary site (if history of malignancy), exposure to cytotoxic cancer therapy for the MSK and Korea Consortia. Summary statistics for a fixed effects meta-analysis are shown.

FIG. 7. Frequency of genes with non-driver mutations among individuals with severe Covid-19.

### DETAILED DESCRIPTION OF THE INVENTION

The main embodiments of the present invention are described in the Summary of the Invention section above, as well as in the Examples, Figures, Brief Description of the Figures, and Claims section of this patent disclosure. Certain additional embodiments and additional details and definitions are provided this Detailed Description of the Invention. It should be understood that variations and combinations of each of the embodiments and details of the invention described above and/or elsewhere in the patent disclosure are contemplated and are intended to fall within the scope of the present invention. The sub-headings provided below, and throughout this patent disclosure, are not intended to denote limitations of the various aspects or embodiments of the invention, which are to be understood by reference to the specification as a whole. For example, this Detailed Description is intended to read in conjunction with, and to expand upon, the description provided in the Summary of the Invention section of this application.

Various terms are defined below. Additional terms are defined elsewhere in this patent disclosure, where used. Terms that are not specifically defined herein may be more fully understood in the context in which the terms are used and/or by reference to the specification in its entirety. Where no explicit definition is provided all technical and scientific terms used herein have the meanings commonly understood by those of ordinary skill in the art to which this invention pertains.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Système International de Unites (SI) accepted form.

Numeric ranges provided herein are inclusive of the numbers defining the range. Where a numeric term is preceded by “about,” the term includes the stated number and values ±10% of the stated number.

Wherever embodiments are described with the language “comprising,” otherwise analogous embodiments described in terms of “consisting of” and/or “consisting essentially of” are included.

As used herein the abbreviation “ARDS” refers to acute respiratory distress syndrome.

As used herein the term “COVID-19” refers to the disease caused by infection with the SARS-CoV-2 coronavirus (including variants thereof) that was declared to have reached pandemic status by the World Health Organization in March 2020.

As used herein the term “severe COVID-19” refers to disease caused by infection with the SARS-CoV-2 coronavirus that results in hypoxia requiring treatment with supplemental oxygen (e.g., supplemental oxygen device>1 L or hypoxia<94%), ARDS, or CRS.

As used herein the abbreviation “CH” refers to clonal hematopoiesis, also called clonal hematopoiesis of indeterminate potential.

As used herein the abbreviation “CHIP” refers to clonal hematopoiesis of indeterminate potential.

As used herein the abbreviation “CRS” refers to cytokine release syndrome.

As used herein the term “molecular analysis” refers to a methodology that can be used to assess the presence and/or quantity and/or frequency of a given nucleic acid molecule or nucleic acid sequence, such as, for example, to assess the presence and/or quantity and/or frequency of a mutant allele in RNA or DNA. Such methodologies include, but are not limited to those involving nucleic acid sequencing (e.g., high throughput next generation sequencing (“NGS”)), bioinformatic analysis of nucleic acid sequences, the polymerase chain reaction (“PCR”) (e.g., quantitative PCR), detection of binding of nucleic acid probes (e.g., various hybridization based techniques), and the like.

As used herein the abbreviation “VAF” refers to variant allele frequency. In the methods described herein, the frequency of a given variant/mutant allele (i.e., the VAF) is typically determined by: (a) obtaining high throughput next generation sequencing data from a sample obtained from a subject (e.g. a sample of cell free DNA or a sample of DNA from circulating leukocytes) and (b) determining from the sequence data the number of sequence reads that have the variant allele, dividing the number sequence reads having the variant allele by the total number of sequence reads, and multiplying the result by 100—to represent the frequency of that variant allele (i.e. the VAF) as a percentage. Methods for performing NGS, identifying/calling variant reads, and calculating VAFs are known in the art. Indeed, algorithms and code for performing such methods are publicly available. For example, suitable methods and code for VAF determination are described in Strom, “Current practices and guidelines for clinical next-generation sequencing oncology testing,” Cancer Biol. Med. 2016 March; 13(1):3-11; Sallman & Padron, “Integrating mutation variant allele frequency into clinical practice in myeloid malignancies,” Hematol. Oncol. Stem Cell Ther. 2016 September; 9(3):89-95; Zehir A, et al., “Mutational landscape of metastatic cancer revealed from prospective clinical sequencing of 10,000 patients,” Nat. Med. 2017, vol. 23(6), pp. 703-713, and Bolton et al., “Cancer therapy shapes the fitness landscape of clonal hematopoiesis.” Nat Genet. 2020 November; 52(11):1219-1226—the contents of which are hereby incorporated by reference, as well as in the Examples section of this patent disclosure.

Several of the embodiments of the present invention involve active agents (sometimes referred to herein as therapeutic agents or therapies). In some embodiments the active agents are anti-cytokine agents. In some embodiments the anti-cytokine agent is an IL-6 inhibitor. In some embodiments the anti-cytokine agent is selected from the group consisting of tocilizumab, siltuximab, anakinra, canakinumab, rilonacept, rituximab, alemtuzumab, ruxolitinib, fedratinib, pacritinib, tofacitinib, tadekinig-alpha, emapalumab, infliximab, etanercept, ronatinib, and corticosteroids. In some embodiments the active agents are anti-COVID-19 antibodies or combinations of antibodies (i.e., antibody cocktails) agents. In some embodiments the anti-COVID-19 antibodies or antibody cocktails are, or comprise, neutralizing antibodies against the spike protein of the SARS-CoV-2 virus. In some embodiments the anti-COVID-19 antibodies or antibody cocktails are, or comprise, bamlanivimab, etesevimab, casirivimab and/or imdevimab. Manufacturers' instructions, product inserts, prescribing information documents, published literature and/or clinical trial information relating to any of such active agents can be referenced in carrying out the present invention and are hereby incorporated by reference in their entireties.

As used herein, the terms “treat,” “treating,” and “treatment” encompass achieving, and/or performing a method that achieves, a detectable improvement in one or more clinical indicators or symptoms associated with the infectious disease (e.g. COVID-19). For example, such terms include, but are not limited to, alleviating, abating, ameliorating, relieving, reducing, inhibiting, preventing, or slowing at least one clinical indicator or symptom of the infectious disease , preventing additional clinical indicators or symptoms of the infectious disease , reducing or slowing the progression of one or more clinical indicators or symptoms of the infectious disease, causing regression of one or more clinical indicators or symptoms of the infectious disease, and the like. In preferred embodiments the treatments described herein reduce, inhibit, prevent or slow the development of cytokine release syndrome and/or sepsis and/or ARDS. As used herein the terms “treat,” “treating,” and “treatment” encompass both preventive/prophylactic treatments and therapeutic treatments. In the case of prophylactic treatments, the methods and compositions provided herein can be used preventatively in subjects that do not yet exhibit any clear or detectable clinical indicators or symptoms of the infectious diseases (such as COVID-19) but that are believed to be at risk of developing such symptoms, for example due to a known infection with the infectious agent (e.g. SARS-Cov-2) or contact with an individual infected with the infectious agent (e.g. SARS-Cov-2). In the case of therapeutic treatments, the methods and compositions provided herein can be used in subjects that are known to be infected and already exhibit one or more clinical indicators or symptoms of the infection. In preferred embodiments the methods and compositions provided herein are used to treat subjects who are known to be infected with the infectious agent but who have not yet developed any serious and/or life-threatening symptoms and/or complications of the infection such as CRS and/or sepsis and/or ARDS. In some such embodiments the subject may exhibit one or more mild symptoms of the infection but have not yet developed any serious and/or life-threatening symptoms and/or complications.

As used herein the term “subject” encompasses all mammalian species, including, but not limited to, humans, non-human primates, dogs, cats, rodents (such as rats, mice and guinea pigs), cows, pigs, sheep, goats, horses, and the like—including all mammalian animal species used in animal husbandry, as well as animals kept as pets and in zoos, etc. In preferred embodiments the subjects are human.

As used herein the term “effective amount” refers to an amount of the specified active agent that is sufficient to achieve, or contribute towards achieving, one or more desirable clinical outcomes, such as those described in the “treatment” description above. In some embodiments, for example where one of the active agents has already been approved for clinical use, the amount of an active agent that is effective may be known in the art. In some embodiments an appropriate “effective” amount may be determined using standard techniques known in the art, such as dose escalation studies, and may be determined taking into account such factors as the desired route of administration, desired frequency of dosing, duration of dosing, etc. Furthermore, an “effective amount” may be determined in the context of any co-administration method to be used. One of skill in the art can readily perform such dosing studies (whether using single agents or combinations of agents) to determine appropriate doses to use. For example, in some embodiments the dose of an active agent of the invention may be calculated based on studies in humans or other mammals carried out to determine efficacy and/or effective amounts of the active agent. The dose may be determined by methods known in the art and may depend on factors such as pharmaceutical form of the active agent, route of administration, whether only one active agent is used or multiple active agents (for example, the dosage of a first active agent required may be lower when such agent is used in combination with a second active agent), and patient characteristics including age, body weight or the presence of any medical conditions affecting drug metabolism.

In some embodiments one or more of the active agents is used at approximately its maximum tolerated dose, for example as determined in phase I clinical trials and/or in dose escalation studies. In some embodiments one or more of the active agents is used at about 90% of its maximum tolerated dose. In some embodiments one or more of the active agents is used at about 80% of its maximum tolerated dose. In some embodiments one or more of the active agents is used at about 70% of its maximum tolerated dose. In some embodiments one or more of the active agents is used at about 60% of its maximum tolerated dose. In some embodiments one or more of the active agents is used at about 50% of its maximum tolerated dose. In some embodiments one or more of the active agents is used at about 50% of its maximum tolerated dose. In some embodiments one or more of the active agents is used at about 40% of its maximum tolerated dose. In some embodiments one or more of the active agents is used at about 30% of its maximum tolerated dose.

In carrying out the treatment methods described herein, any suitable method or route of administration can be used to deliver the active agents described herein. In some embodiments systemic administration may be employed. In some embodiments oral administration may be employed. In some embodiments intravenous administration may be employed. In some embodiments subcutaneous administration may be employed.

In certain embodiments the compositions and methods of treatment provided herein may be employed together with other compositions and treatment methods useful for treatment of severe infection (such as severe COVID-19), including, compositions and methods useful for respiratory support, such as supply of oxygen, artificial ventilation, and the like. Similarly, in certain embodiments the methods of treatment provided herein may be employed together with procedures used to monitor infectious disease status/progression.

In some embodiments the treatment methods described herein may be employed in conjunction with, or may involve performing, a assay to determine if the subject has an infectious disease (such as COVID-19). Examples of suitable assays include those based on performing viral culture, performing PCR or a similar assay to nucleic acids of the infectious agent, performing an assay to an antigen of the infectious agent, performing an assay to detect an antibody that binds to an antigen of the infectious agent, and the like. Several of such assays (which may be referred to as “diagnostic tests” or “diagnostic assays”) are known in the art and can be employed in the present invention.

In some embodiments the treatment methods described herein may be employed in conjunction with, or may involve performing, an assay (which may be referred to as a “diagnostic test” or “diagnostic assay”) to determine if the subject has CH and/or a CH-related mutation (such as a Tet2 mutation) and/or to determine the frequency of a CH-associated variant allele/acquired mutation. Several of such assays and methodologies are known in the art and described in the published literature and can be employed in the context of and/or referred to in carrying out the present invention. In addition suitable assays and methodologies are described in Example 1 of this patent disclosure, below.

The present invention is further described with reference to the following non-limiting Examples.

### EXAMPLES

### Example 1

### Clonal Hematopoiesis is Associated with Risk of Severe Covid-19

Acquired mutations that lead to clonal expansion are common in the normal aging hematopoietic system (clonal hematopoiesis, or CH), yet are known to alter stem/progenitor and lymphoid function and response to environmental stressors, including systemic infections^{5,6,9,10}. The mutational events that drive CH overlap with known drivers of hematologic malignancies. However, the majority of mutations in CH appear to occur outside of canonical cancer driver genes^{11,12}. The impact of individual mutational events on hematopoietic stem and progenitor cells differs by the nature of the genomic aberration. For example, chromosomal aneuploidies result in a predisposition for lymphoid fate specification and transformation^{13,14} while point mutations in DNMT3A result in increased myeloid differentiation^{6,15}. Heterogeneity also exists across CH phenotypes by driver gene with regards to impact on inflammatory signaling⁶. For example, mutations in TET2 result in heightened secretion of several cytokines including IL-1β/IL-6 signaling that may partially explain the increased risk of cardiovascular disease^{5,9,16}. Moreover, systemic infections and the resultant inflammatory signals can lead to increased clonal fitness of TET2 mutant cells and clonal expansion^{10,17,18}.

We hypothesized that there may be a relationship between CH and COVID-19—including the potential for an association of CH with increased COVID-19 disease severity. To the best of our knowledge, the possibility of relationship between CH, infectious risk and/or infectious disease severity has not been previously studied or demonstrated.

The study described herein includes patients from two separate cohorts. The first cohort was composed of patients with solid tumors treated at Memorial Sloan Kettering Cancer Center (MSK) with blood previously sequenced using MSK-IMPACT, a previously validated targeted gene panel capturing all commonly mutated CH-associated genes²⁴. Of these patients, 1,626 were tested for SARS-CoV-2 (the virus that causes Covid-19) RNA; 403 (24.8%) of individuals tested positive for SARS-CoV-2. The second cohort included 112 previously healthy individuals without cancer who were hospitalized for COVID-19 at hospitals in South Korea (KoCH cohort). The KoCH cohort was sequenced using a targeted next generation sequencing (NGS) panel from Agilent (89 genes) which was designed to include commonly occurring CH genes.

For both cohorts, the primary outcome was severe COVID-19 infection, defined as the presence of hypoxia requiring supplemental oxygen (oxygen device>1 L or hypoxia<94%).

Multivariable logistic regression analysis was used, adjusting for covariates including age, smoking, prior COVID-19 related comorbidities, and prior cancer treatment to determine the association between severe COVID-19 and CH in each population. A fixed-effects meta-analysis was performed to estimate the association in the overall population. The full statistical rationale is further described in the Methods section.

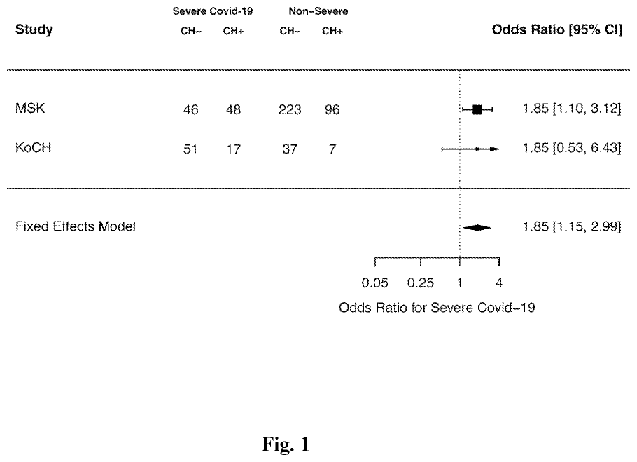

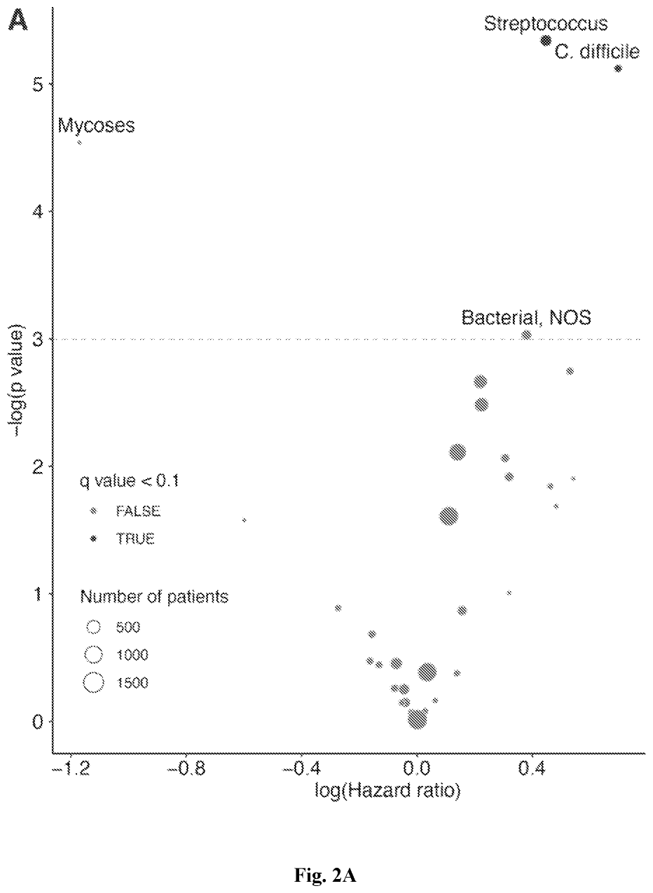

Among COVID-19 positive individuals, 23% (N=94) and 61% (N=68) had severe disease in the MSK and KoCH cohorts, respectively. Overall, CH was observed in 35% of Covid-19 positive cases at MSK and 21% in KoCH. Of note, when restricting the MSK-IMPACT panel to the 89 genes included in the KoCH panel, 20% of COVID-19 positive cases at MSK had CH. In the MSK cohort, CH was observed in 51% and 30% of patients with severe versus non-severe Covid-19, respectively (adjusted OR: 1.85, 95% CI 1.10-3.12, FIG. 1). In the KoCH cohort, CH was observed in 25% and 15.9% of patients with severe versus non-severe Covid-19, respectively (adjusted OR 1.85, 95% CI 0.53-6.43, FIG. 1). In a fixed effects meta-analysis of odds-ratio estimates from the multivariable logistic regression models employed in each separate cohort analysis, the presence of CH was associated with an increased risk of severe COVID-19 (OR=1.85, 95%=1.15-2.99, p=0.01) (FIG. 1).

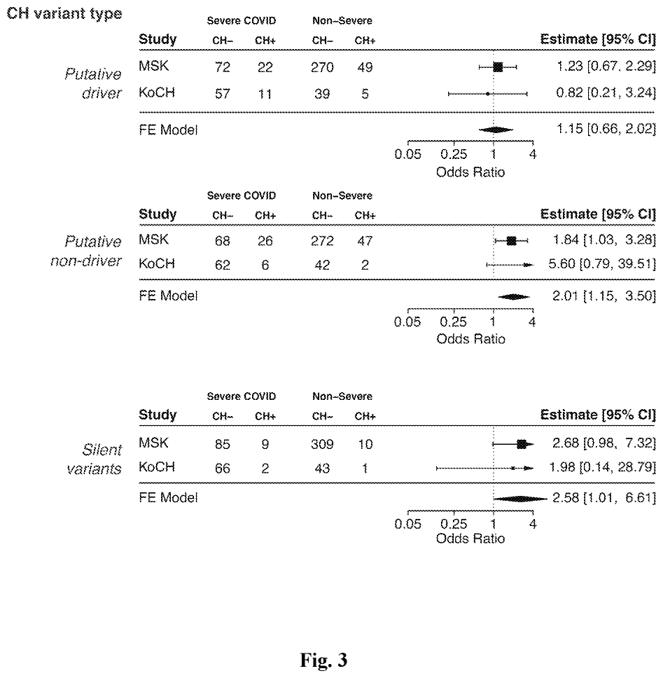

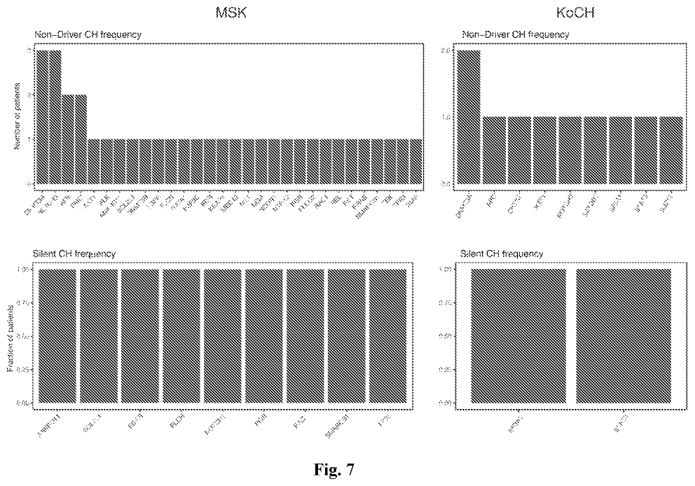

Using previously described methods²⁴, CH mutations were classified as known or hypothesized cancer putative drivers (PD-CH) or non-putative drivers (non-PD CH). The majority of CH mutations were classified as PD-CH (52% in the MSK cohort and 67% in the KoCH dataset). To explore the association between particular mutation types and Covid-19 severity, a stratified analysis of COVID-19 severity by PD-CH versus non-PD CH status was performed. A significant association was observed between non-PD CH and severe Covid-19 (OR=2.01, 95% CI=1.15-3.50, p=0.02), as well as between silent (synonymous) CH and severe COVID-19 (OR=2.58, 95% CI 1.01-6.61, p=0.05). There was not a statistically-significant association between PD-CH and severe COVID-19 infection (OR=1.15, 95% CI=0.61-2.02, p=0.77: FIG. 3). Most non-PD mutations in COVID-19 positive cases occurred in non-recurrently mutated genes (65% at MSK and 76.9% in KoCH, FIG. 7).

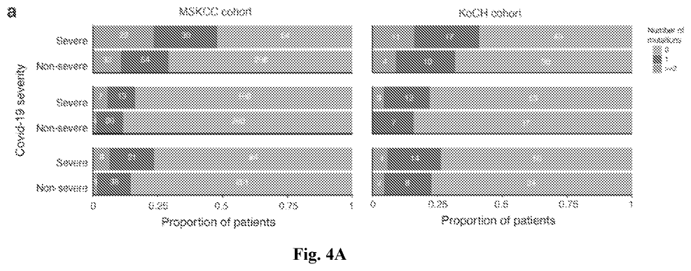

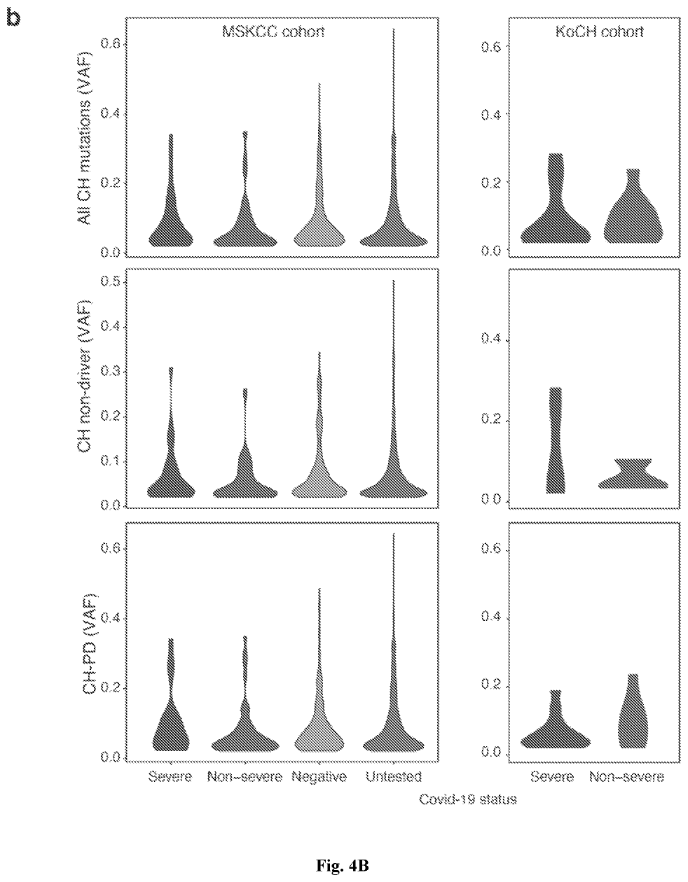

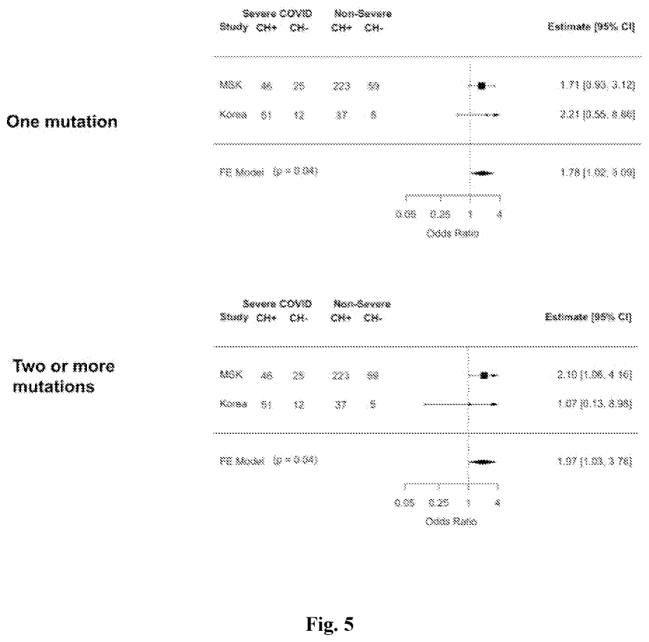

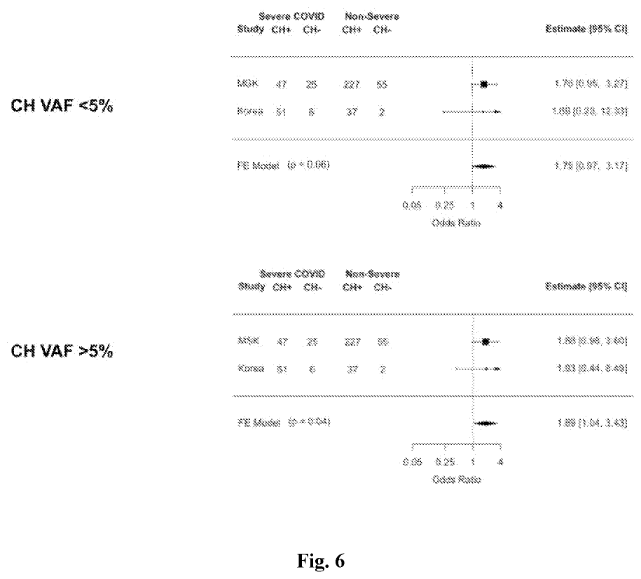

The strength of the association between CH and severe COVID-19 was similar among patients with one CH mutation (OR=1.78, 95% CI=1.0-3.1, p=0.04) and multiple CH mutations (OR=2.0, 95% CI=1.0-3.8, p=0.04). Patients with a maximum CH variant allele frequency (VAF) of >5% showed a significant association with severe COVID-19 (OR=1.9, 95% CI=1.0-3.4, p=0.04). This association trended towards statistical significance in patients with any CH mutation and a maximum VAF<5% (OR=1.75, 95% CI=0.97-3.17, p=0.06: FIGS. 4-6). These data suggest that the presence of CH and resultant alterations in hematopoietic differentiation, and not specific mutant alleles, is predictive of COVID-19 disease severity.

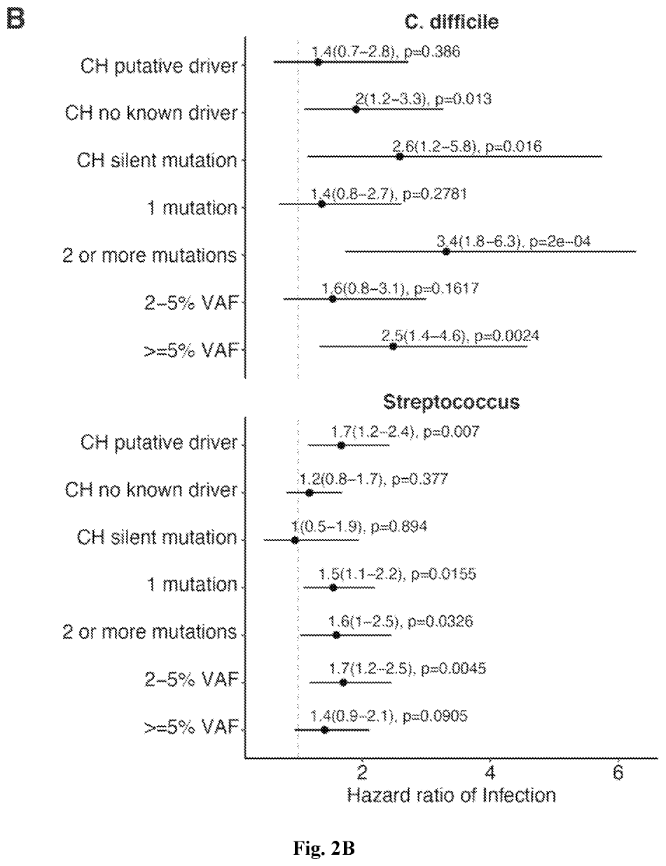

Studies were also performed to explore the relationship between CH and other types of infections. Billing codes from 14,211 solid tumor patients treated at MSK who underwent blood sequencing by MSK-IMPACT were analyzed. Using a previously established phenome-wide-association study (Phe-WAS) methodology²⁵, patient billing codes were mapped to categories of infectious disease. Multivariable Cox proportional hazards regression was used to estimate the hazard ratio (HR) for risk of infection among CH positive compared to CH negative individuals. Given the number of model covariates, the analysis was limited to 32 infection subclasses that affected at least 80 individuals (see Methods). Multiple infection types were associated with CH, although many associations were not statistically-significant after multiplicity adjustment (FIG. 2A). CH was significantly (FDR-corrected p-value<0.10) associated with the onset of two infection subclasses: Clostridium difficile infection (HR=2.0, 95% CI: 1.2-3.3, p=6×10−3) and Streptococcus/Enterococcus infection (HR=1.5, 95% CI=1.1-2.1, p=5×10−3). When stratified by CH-mutation characteristics, patients with two or more CH-mutations had a stronger association with Clostridium difficile infection (OR=3.4, 95% CI=1.8-6.3, p=2×10−4) compared to patients with one CH-mutation (OR=1.4, 95% CI=0.8-2.7, p=0.28). The association between CH and Clostridium difficile infection was significant for mutations with a VAF of >5% (OR=2.5, 95% CI=1.4-4.6, p=0.002) but not mutations with a VAF of 2-5% (OR=1.6, 95% CI=0.8-3.1, p=0.17). Similar to COVID-19 severity, the association between CH and Clostridium difficile infection was significant for non-PD CH (OR=2.0, 95% CI=1.2-3.3, p=0.01) and silent mutations (OR=2.6, 95% CI=1.2-5.8, p=0.02) but not CH-PD (OR=1.4, 95% CI=0.7-2.8, p=0.39) (FIG. 2B).

In summary, the results of this study show that in cancer and non-cancer patients CH is associated with increased COVID-19 severity. In a large cancer patient cohort, CH is also associated with other severe infections, namely Streptococcus/Enterococccus and Clostridium difficile infections. The hematopoietic system is a key regulator of inflammation and immunity. A substantial body of evidence now links somatic alterations in hematopoietic stem and progenitor cells to a variety of health outcomes, with inflammation emerging as a key mediator.^{2-5,10-13} The data provided here demonstrates an association between CH and increased infection severity.

Methods

Sample Ascertainment and Clinical Data Extraction

The study population included 9,307 patients with non-hematologic cancers at MSKCC who underwent matched tumor and blood sequencing using the MSK-IMPACT panel on an institutional prospective tumor sequencing protocol (ClinicalTrials.gov number, NCT01775072). Subjects who had a hematologic malignancy diagnosed after MSK-IMPACT testing or who had an active hematologic malignancy at the time of blood draw were excluded. Demographics, smoking history, exposure to oncologic therapy and primary tumor site were extracted from the electronic health record. Accuracy of populated information was manually checked by three independent physicians. The presence of co-existing medical comorbidities known to correlate with COVID-19 severity including diabetes, COPD, asthma, hypertension and cardiovascular disease, were ascertained. SARS-CoV-2 status was determined using RT-PCR. Severe COVID-19 was defined as the presence of hypoxia requiring supplemental oxygen (supplemental oxygen device>1 L or hypoxia<94%) resulting from COVID-19 infection. There were seven subjects with COVID-19 for whom there was minimal documentation of clinical course following COVID-19 infection and these individuals were excluded. There were three individuals with metastatic cancer and progression of disease at the time of COVID-19 where it was unclear whether documented hypoxia could be attributed to COVID-19 or disease progression. These subjects were also excluded.

Laboratory-confirmed patients with COVID-19 in four hospitals in the Republic of Korea were approached for consent to this study. Blood was drawn following confirmation of Covid-19 positivity. Clinical and laboratory characteristics were retrospectively reviewed using the electronic medical record systems of each institution. Hypoxia requiring supplemental oxygen was defined as supplemental oxygen device>1 L with O2<94% resulting from COVID-19 infection. Subjects who had an active malignancy at the time of blood draw were excluded.

Sequencing and Variant Calling

Subjects in the MSK cohort had a tumor and blood sample (as a matched normal control) sequenced using MSK-IMPACT, an FDA-authorized hybridization capture-based next-generation sequencing assay encompassing all protein-coding exons from the canonical transcript of 341, 410, or 468 cancer-associated genes. MSK-IMPACT is validated and approved for clinical use by New York State Department of Health Clinical Laboratory Evaluation Program. The sequencing test utilizes genomic DNA extracted from formalin fixed paraffin embedded (FFPE) tumor tissue as well as matched patient blood samples. DNA is sheared and DNA fragments are captured using custom probes⁴⁷. MSK-IMPACT contains most of the commonly reported CH genes with the exception that earlier versions of the panel did not contain PPM1D or SRSF2. Pooled libraries were sequenced on an Illumina HiSeq 2500 with 2×100 bp paired-end reads. Sequencing reads were aligned to the human genome (hg19) using BWA (0.7.5a). Reads were re-aligned around indels using ABRA (0.92), followed by base quality score recalibration with Genome Analysis Toolkit (GATK) (3.3-0). Median coverage in the blood samples was 497×, and median coverage in the tumors was 790×. Variant calling for each blood sample was performed unmatched, using a pooled control sample of DNA from 10 unrelated individuals as a comparator. Single nucleotide variants (SNVs) were called using Mutect and VarDict. Insertions and deletions were called using Somatic Indel Detector (SID) and VarDict. Variants that were called by two callers were retained. Dinucleotide substitution variants (DNVs) were detected by VarDict and retained if any base overlapped a SNV called by Mutect. All called mutations were genotyped in the patient-matched tumor sample. Mutations were annotated with VEP (version 86) and OncoKb. A series of post-processing filters were applied to further remove false positive variants caused by sequencing artifacts and putative germline polymorphisms as previously described²⁴.

Blood-derived DNA from the KoCH cohort was sequenced using a panel of 89 genes frequently mutated in CH. All NGS libraries were prepared using the Agilent SureSelect XT HS and XT Low input enzymatic fragmentation kit. Pooled Libraries were sequenced on an Illumina NovaSeq6000 with 2×150 bp paired-end reads. Sequencing reads were trimmed with SeqPrep (v0.3) and Sickle (v1.33) and aligned to the human genome (hg19) using BWA-MEM (v0.7.10). PICARD (v1.94) was used for duplicate marking followed by indel realignment and base quality score recalibration with GATK light (v2.3.9). The mean depth of coverage of samples was higher than 800×. Variant calling was performed using SNver (v0.4.1), LoFreq (v0.6.1), GATK UnifiedGenotyper (v2.3.9) for SNVs. For Insertions and deletions an InDel caller was used²⁶. The union of all called results were filtered meeting the criteria (total reads>=10, Alt reads>=10, positive Alt reads>=5, negative Alt reads>=5, MQV>=30, BQV>=30) and VAF falling between 2% and 30%. Common germline variants were filtered based on genomAD, 1k Genome v3, ESP6500 and ExAC data. Lastly, technical artifact calls with maf>2% were filtered based on an internal panel of 1000 individuals who were CH negative.

Variant Annotation

Variants from the MSK and KoCH cohort were uniformly annotated according to evidence for functional relevance in cancer (putative driver or CH-PD). Variants were annotated as oncogenic if they fulfilled any of the following criteria: 1) truncating variants in NF1, DNMT3A, TET2, IKZF1, RAD21, WT1, KMT2D, SH2B3, TP53, CEBPA, ASXL1, RUNX1, BCOR, KDM6A, STAG2, PHF6, KMT2C, PPM1D, ATM, ARID1A, ARID2, ASXL2, CHEK2, CREBBP, ETV6, EZH2, FBXW7, MGA, MPL, RB1, SETD2, SUZ12, ZRSR2 or in CALR exon 9; 2) any truncating mutations (nonsense, essential splice site or frameshift indel) in known tumor suppressor genes as per the Cancer Gene Census, OncoKB, or the scientific literature; 3) translation start site mutations in SH2B3; 4) TERT promoter mutations; 5) FLT3-ITDs; 6) in-frame indels in CALR, CEBPA, CHEK2, ETV6, EZH2; 7) any variant occurring in the COSMIC “haematopoietic and lymphoid” category greater than or equal to 10 times; 8) any variant reported as somatic at least 20 times in COSMIC; 9) any variant noted as potentially oncogenic in an in-house dataset of 7,000 individuals with myeloid neoplasm greater than or equal to 5 times; 10) any loci (defined by the amino acid location) reported as having at least 5 missense mutations and at least one exact mutational match in TopMed6.

Statistical Analysis

CH and COVID-19 Severity

Multivariable logistic regression was used to evaluate for an association between clonal hematopoiesis and COVID-19 severity adjusting for age (measured as a continuous variable), gender, race, smoking history and co-existing medical comorbidities including diabetes, COPD/asthma and cardiovascular disease. This was done separately for the MSK and KoCH cohorts. For solid tumor patients at MSK adjustments were also made for primary tumor site (thoracic or non-thoracic cancer) and receipt of cytotoxic chemotherapy before and after IMPACT blood draw. A fixed effects meta-analysis of the MSK and KoCH cohorts was performed to jointly estimate the odds ratio for severe COVID-19 among CH positive compared to CH negative individuals.

CH and Risk of Infection in the MSK Cohort

Billing codes from 14,211 solid tumor patients at MSKCC who had their blood sequenced using MSK-IMPACT were analyzed. The phecode nomenclature developed at Vanderbilt⁹ was used to map billing codes to infectious disease subtypes. Subjects who were billed using a ICD9/10 code within the phecode for the first time following their sequencing blood draw with evidence of CH were considered to have an incident infection. Those who were billed for an ICD9/10 code within the phecode prior to blood draw were removed from the analysis of that phecode. In order to evaluate the accuracy of the billing code data, the presence of a documented Clostridium Difficile or Streptococcus infection in an EMR physician note was manually checked for patients respectively identified by billing codes (N=525 patients) by three independent physicians using criteria for infection onset. Billing codes were highly accurate in identifying the presence of the respective infectious disease (concordance>95%).

Cox proportional hazards regression was used to estimate the hazard ratio for risk of infection among those with CH compared to CH negative individuals. The date of blood draw (used for MSK-IMPACT sequencing) served as the onset date for this time-to-event analysis; the end-date was the date of billing code entry for the infectious disease subtype phecode, death or last follow-up, whichever came first. All models were adjusted for age, gender, race, smoking, tumor type, and cumulative exposure to cytotoxic chemotherapy prior to blood draw and after blood draw as previously described¹⁰. Following the 10:1 rule regarding the number of covariates in a multivariable model in proportion to the number of events¹⁶, infection subclasses populated with less than 80 individuals were excluded. The analysis utilized multiplicity correction with the Benjamini-Hochberg method to establish adjusted q-values for hazard ratio with a prespecified false-discovery-rate (FDR)<0.10.

All the statistical analyses were performed with the use of the R statistical package (www.r-project.org).

### Example 2

### Anti-Cytokine or Anti-Inflammatory Therapy for Treatment of Patients with CH

The utility of anti-cytokine or anti-inflammatory therapy for the treatment and/or prevention of potentially life-threatening complications of infection such as sepsis and/or ARDS (including, but not limited to that associated with SARS-Cov-2 infection) in patients with CH is confirmed by performing a prospective clinical trial in which patients with an infection are assessed to determine their CH status and then treated with anti-cytokine or anti-inflammatory therapy. Optionally non-treated control groups are included in the study. The subjects are evaluated for the development of and/or severity of CRS, sepsis, and/or ARDS. It is expected that in those patients having CH, treatment with the anti-cytokine or anti-inflammatory therapy will reduce the development of and/or severity of CRS, sepsis and/or ARDS to a greater extent than in subjects that are treated similarly but that do not have CH.

As an alternative to, or in addition to, the above prospective clinical trial, a retrospective analysis of previously performed clinical trials of anti-cytokine or anti-inflammatory therapies is performed to assess the CH status of patients with infectious diseases treated with anti-cytokine or anti-inflammatory therapies. It is expected that the occurrence of and/or severity of potentially life threatening complications of infection such as sepsis and/or ARDS (including, but not limited to that associated with SAR-Cov-2 infection) will be significantly reduced in those patients that have CH as compared to those patients that do not have CH.

### Example 3

The utility of anti-COVID-19 antibody therapy for the treatment and/or prevention of potentially life threatening complications of infection such as ARDS associated with SARS-Cov-2 infection in patients with CH is confirmed by performing a prospective clinical trial in which patients with a SARS-Cov-2 infection, or at risk of SARS-Cov-2 infection, are assessed to determine their CH status and then treated with an anti-COVID-19 antibody therapy, such as neutralizing antibodies against the spike protein of SARS-CoV-2, or cocktails of such antibodies. Optionally non-treated control groups are included in the study. The subjects are then evaluated for the development of and/or severity of CRS and/or ARDS. It is expected that in those patients having CH, treatment with the anti-COVID-19 antibody therapy will reduce the development of and/or severity of CRS and/or ARDS as compared to that observed in subjects that are treated similarly but that do not have CH.

As an alternative to, or in addition to, performing the above prospective clinical trial, a retrospective analysis of previously performed clinical trials of anti-COVID-19 antibody therapies can be performed. It is expected that, if a retrospective analysis is performed to assess the CH status of patients with infectious diseases treated with anti-COVID-19 antibody therapies, the occurrence of and/or severity of potentially life threatening complications of infection such as CRS and/or ARDS will be significantly reduced in those patients that have CH as compared to those patients that do not have CH.

The following references are incorporated by reference herein in their entirety:

### REFERENCES

1. Genovese, G. et al. Clonal Hematopoiesis and Blood-Cancer Risk Inferred from Blood DNA Sequence. N. Engl. J. Med. 371, 2477-2487 (2014).
2. Jaiswal, S. et al. Age-Related Clonal Hematopoiesis Associated with Adverse Outcomes. N. Engl. J. Med. 371, 2488-2498 (2014).
3. Abelson, S. et al. Prediction of acute myeloid leukaemia risk in healthy individuals. Nature 559, 400-404 (2018).
4. Desai, P. et al. Somatic mutations precede acute myeloid leukemia years before diagnosis. Nat. Med. 24, 1015-1023 (2018).
5. Bick Alexander G. et al. Genetic Interleukin 6 Signaling Deficiency Attenuates Cardiovascular Risk in Clonal Hematopoiesis. Circulation 141, 124-131 (2020).
6. Bick, A. G. et al. Inherited causes of clonal haematopoiesis in 97,691 whole genomes. Nature 1-7 (2020) doi:10.1038/s41586-020-2819-2.
7. Del Valle, D. M. et al. An inflammatory cytokine signature predicts COVID-19 severity and survival. Nat. Med. 26, 1636-1643 (2020).
8. Zhou, F. et al. Clinical course and risk factors for mortality of adult inpatients with COVID-19 in Wuhan, China: a retrospective cohort study. The Lancet 395, 1054-1062 (2020).
9. Sano, S. et al. Tet2-Mediated Clonal Hematopoiesis Accelerates Heart Failure Through a Mechanism Involving the IL-1β/NLRP3 Inflammasome. J. Am. Coll. Cardiol. 71, 875-886(2018).
10. Cai, Z. et al. Inhibition of Inflammatory Signaling in Tet2 Mutant Preleukemic Cells Mitigates Stress-Induced Abnormalities and Clonal Hematopoiesis. Cell Stem Cell 23, 833-849.e5 (2018).
11. Zink, F. et al. Clonal hematopoiesis, with and without candidate driver mutations, is common in the elderly. Blood 130, 742-752 (2017).
12. Poon, G., Watson, C. J., Fisher, D. S. & Blundell, J. R. Synonymous mutations reveal genome-wide driver mutation rates in healthy tissues. bioRxiv 2020.10.08.331405 (2020) doi:10.1101/2020.10.08.331405.
13. Loh, P.-R. et al. Insights into clonal haematopoiesis from 8,342 mosaic chromosomal alterations. Nature 559, 350-355 (2018).
14. Terao, C. et al. Chromosomal alterations among age-related haematopoietic clones in Japan. Nature 584, 130-135 (2020).
15. Coombs, C. C. et al. Identification of Clonal Hematopoiesis Mutations in Solid Tumor Patients Undergoing Unpaired Next-Generation Sequencing Assays. Clin. Cancer Res. Off. J. Am. Assoc. Cancer Res. 24, 5918-5924 (2018).
16. Jaiswal, S. et al. Clonal hematopoiesis and risk for atherosclerotic cardiovascular disease. N. Engl. J. Med. 377, 111-121 (2017).
17. Meisel, M. et al. Microbial signals drive pre-leukaemic myeloproliferation in a Tet2-deficient host. Nature 557, 580-584 (2018).
18. Zeng, H. et al. Antibiotic treatment ameliorates Ten-eleven translocation 2 (TET2) loss-of-function associated hematological malignancies. Cancer Lett. 467, 1-8 (2019).
19. Jee, J. et al. Chemotherapy and COVID-19 Outcomes in Patients With Cancer. J. Clin. Oncol. 38, 3538-3546 (2020).
20. Guan, W. et al. Clinical Characteristics of Coronavirus Disease 2019 in China N. Engl. J. Med. 382, 1708-1720 (2020).
21. Livingston, E. & Bucher, K. Coronavirus Disease 2019 (COVID-19) in Italy. JAMA 323, 1335(2020).
22. Petrilli, C. M. et al. Factors associated with hospital admission and critical illness among 5279 people with coronavirus disease 2019 in New York City: prospective cohort study. BMJ 369, (2020).
23. Sung, H. K. et al. Clinical Course and Outcomes of 3,060 Patients with Coronavirus Disease 2019 in Korea, January-May 2020. J. Korean Med. Sci. 35, e280 (2020).
24. Bolton, K. L. et al. Cancer therapy shapes the fitness landscape of clonal hematopoiesis. Nat. Genet. 52, 1219-1226 (2020).
25. Wu, P. et al. Mapping ICD-10 and ICD-10-CM Codes to Phecodes: Workflow Development and Initial Evaluation. JMIR Med. Inform. 7, (2019).
26. Phi, J. H. et al. NPM1 as a potential therapeutic target for atypical teratoid/rhabdoid tumors. BMC Cancer 19, 848 (2019).
27. Bolton K L, Zehir A, Ptashkin R N, Patel M, Gupta D, Sidlow R, Papaemmanuil E, Berger M F, Levine R L. The Clinical Management of Clonal Hematopoiesis: Creation of a Clonal Hematopoiesis Clinic. Hematol Oncol Clin North Am. 2020 April; 34(2):357-367. doi: 10.1016/j.hoc.2019.11.006. Epub 2020 Jan. 18. Review. PMID: 32089215
28. Zhang Q, Zhao K, Shen Q, Han Y, Gu Y, Li X, Zhao D, Liu Y, Wang C, Zhang X, Su X, Liu J, Ge W, Levine R L, Li N, Cao X. Tet2 is required to resolve inflammation by recruiting Hdac2 to specifically repress IL-6. Nature. 2015 Sep. 17; 525(7569):389-393. doi: 10.1038/nature15252. Epub 2015 Aug. 19
29. Sano S, Oshima K, Wang Y, MacLauchlan S, Katanasaka Y, Sano M, Zuriaga M A, Yoshiyama M, Goukassian D, Cooper M A, Fuster J J, Walsh K. Tet2-Mediated Clonal Hematopoiesis Accelerates Heart Failure Through a Mechanism Involving the IL-1β/NLRP3 Inflammasome. J Am Coll Cardiol. 2018 Feb. 27; 71(8):875-886. doi: 10.1016/j.jacc.2017.12.037.
30. Henderson L A, Canna S W, Schulert G S, Volpi S, Lee P Y, Kernan K F, Caricchio R, Mahmud S, Hazen Halyabar O, Hoyt K J, Han J, Grom A A, Gattorno M, Ravelli A, de Benedetti F, Behrens E M, Cron R Q, Nigrovic P A. On the alert for cytokine storm: Immunopathology in COVID-19. Arthritis Rheumatol. 2020 Apr. 15. doi: 10.1002/art.41285. [Epub ahead of print] PMID: 32293098
31. Chen et al., Detectable serum SARS-CoV-2 viral load (RNAaemia) is closely correlated with drastically elevated interleukin 6 (IL-6) level in critically ill COVID-19 patients. Clin Infect Dis. 2020 Apr. 17. pii: ciaa449. doi:10.1093/cid/ciaa449. [Epub ahead of print]
32. Liu et al., Can we use interleukin-6 (IL-6) blockade for coronavirus disease 2019 (COVID-19)-induced cytokine release syndrome (CRS)? J Autoimmun. 2020 Apr. 10: 102452. doi: 10.1016/j.jaut.2020.102452. [Epub ahead of print]
33. Libby et al., Clonal Hematopoiesis: Crossroads of Aging, Cardiovascular Disease, and Cancer: JACC Review Topic of the Week. J Am Coll Cardiol. 2019 Jul. 30; 74(4):567-577. doi: 10.1016/j.jacc.2019.06.007.
34. Zehir et al., Mutational landscape of metastatic cancer revealed from prospective clinical sequencing of 10,000 patients. Nat Med. 2017 June; 23(6):703-713. doi: 10.1038/nm.4333. Epub 2017 May 8. Erratum in: Nat Med. 2017 Aug. 4; 23 (8):1004. PMID: 28481359; PMCID: PMC5461196.
35. Strom, “Current practices and guidelines for clinical next-generation sequencing oncology testing,” Cancer Biol. Med. 2016 March; 13(1):3-11;
36. Sallman & Padron, “Integrating mutation variant allele frequency into clinical practice in myeloid malignancies,” Hematol. Oncol. Stem Cell Ther. 2016 September; 9(3):89-95.

We claim:

1. A method of treating COVID-19 in a subject in need thereof, the method comprising:
administering an effective amount of an anti-cytokine agent to a subject with COVID-19, wherein the subject has clonal hematopoiesis (CH), thereby treating COVID-19 in the subject.

2. The method of claim 1, further comprising performing an assay to determine if the subject has CH, wherein the assay is performed prior to administering the anti-cytokine agent to the subject.

3. The method of claim 2, wherein the assay comprises performing a molecular analysis.

4. The method of claim 2, wherein the assay comprises performing DNA sequencing.

5. The method of claim 2, wherein the assay comprises performing DNA sequencing of DNA of circulating leukocytes from the subject or cell-free DNA from the subject.

6. The method of claim 2, wherein the assay comprises determining the variant allele frequency of an acquired mutation in a leukemia-associated gene.

7. The method of claim 2, wherein the assay comprises determining the variant allele frequency of an acquired mutation in a leukemia-associated gene in circulating leukocytes or cell-free DNA from the subject.

8. The method of claim 2, wherein the subject is determined to have CH if the subject has a variant allele frequency of ≥about 2 percent of an acquired mutation of a leukemia-associated gene.

9. The method of claim 2, wherein the subject is determined to have CH if the subject has a variant allele frequency of ≥about 2 percent of an acquired mutation of the DNMT3A, TET2, or ASXL1 genes.

10. The method of claim 2, wherein the subject is determined to have CH if the subject has a variant allele frequency of ≥about 2 percent of an acquired mutation of the TET2 gene.

11. The method of claim 1, further comprising performing an assay to determine if the subject is infected with a SARS-CoV-2 virus, wherein the assay is performed prior to administering the anti-cytokine agent to the subject.

12. The method of claim 11, wherein the assay comprises performing viral culture to detect the SARS-CoV-2 virus.

13. The method of claim 11, wherein the assay comprises detecting nucleic acids of the SARS-CoV-2 virus.

14. The method of claim 13, wherein the assay comprises performing PCR.

15. The method of claim 1, wherein the anti-cytokine agent is administered to the subject in the absence of, or prior to the onset of, symptoms of acute respiratory distress syndrome (ARDS) or cytokine release syndrome (CRS).

16. The method of claim 1, wherein the anti-cytokine agent is an IL-6 inhibitor.

17. The method of claim 1, wherein the anti-cytokine agent is selected from the group consisting of tocilizumab, siltuximab, anakinra, canakinumab, rilonacept, rituximab, alemtuzumab, ruxolitinib, fedratinib, pacritinib, tofacitinib, tadekinig-alpha, emapalumab, infliximab, etanercept, ronatinib, and corticosteroids.

18. A method of determining if a subject infected with a SARS-CoV-2 virus is a candidate for initiation of anti-cytokine therapy, the method comprising:
determining if the subject has CH, wherein, if the subject has CH the subject is a candidate for initiation of anti-cytokine therapy.

19. The method of claim 18, wherein the step of determining if the subject has CH is performed in the absence of, or prior to the onset of, symptoms of ARDS or CRS in the subject.

20. The method of claim 18, wherein the subject is determined to have CH if the subject has a variant allele frequency of ≥about 2 percent of an acquired mutation of a leukemia-associated gene.

21. The method of claim 18, wherein the subject is determined to have CH if the subject has a variant allele frequency of ≥about 2 percent of an acquired mutation of a leukemia-associated gene in circulating leukocytes or cell-free DNA from the subject.

22. The method of claim 18, wherein the subject is determined to have CH if the subject has a variant allele frequency of ≥about 2 percent of an acquired mutation of the DNMT3A, TET2, or ASXL1 genes.

23. The method of claim 18, wherein the subject is determined to have CH if the subject has a variant allele frequency of ≥about 2 percent of an acquired mutation of the DNMT3A, TET2, or ASXL1 genes in circulating leukocytes or cell-free DNA from the subject.

24. The method of claim 18, wherein the is determined to have CH if the subject has a variant allele frequency of ≥about 2 percent of an acquired mutation of the TET2 gene.

25. The method of claim 18, wherein the is determined to have CH if the subject has a variant allele frequency of ≥about 2 percent of an acquired mutation of the TET2 gene in circulating leukocytes or cell-free DNA from the subject.

26. The method of claim 18, comprising performing an assay to determine if the subject has CH.

27. The method of claim 26, wherein the assay comprises performing a molecular analysis.

28. The method of claim 26, wherein the assay comprises performing DNA sequencing.

29. The method of claim 26, wherein the assay comprises performing DNA sequencing of DNA of circulating leukocytes from the subject or cell-free DNA from the subject.

30. The method of claim 26, wherein the assay comprises determining the variant allele frequency of an acquired mutation in a leukemia-associated gene.

31. The method of claim 26, wherein the assay comprises determining the variant allele frequency of an acquired mutation in a leukemia-associated gene in circulating leukocytes or cell-free DNA from the subject.

32. The method of claim 26, wherein the assay comprises determining the variant allele frequency of an acquired mutation in the NMT3A, TET2, or ASXL1 gene.

33. The method of claim 26, wherein the assay comprises determining the variant allele frequency of an acquired mutation in the NMT3A, TET2, or ASXL1 gene in circulating leukocytes or cell-free DNA from the subject.

34. The method of claim 26, wherein the assay comprises determining the variant allele frequency of an acquired mutation in the TET2 gene.

35. The method of claim 26, wherein the assay comprises determining the variant allele frequency of an acquired mutation in the TET2 gene in circulating leukocytes or cell-free DNA from the subject.

36. A method of treating COVID-19 in a subject in need thereof, the method comprising: administering an effective amount of an anti-COVID-19 antibody therapy to a subject with COVID-19, wherein the subject has clonal hematopoiesis (CH), thereby treating COVID-19 in the subject.

37. The method of claim 36, further comprising performing an assay to determine if the subject has CH, wherein the assay is performed prior to administering the anti-COVID-19 antibody therapy to the subject.

38. The method of claim 37, wherein the assay comprises performing a molecular analysis.

39. The method of claim 37, wherein the assay comprises performing DNA sequencing.

40. The method of claim 37, wherein the assay comprises performing DNA sequencing of DNA of circulating leukocytes from the subject or cell-free DNA from the subject.

41. The method of claim 37, wherein the assay comprises determining the variant allele frequency of an acquired mutation in a leukemia-associated gene.

42. The method of claim 37, wherein the assay comprises determining the variant allele frequency of an acquired mutation in a leukemia-associated gene in circulating leukocytes or cell-free DNA from the subject.

43. The method of claim 37, wherein the subject is determined to have CH if the subject has a variant allele frequency of ≥about 2 percent of an acquired mutation of a leukemia-associated gene.

44. The method of claim 37, wherein the subject is determined to have CH if the subject has a variant allele frequency of ≥about 2 percent of an acquired mutation of the DNMT3A, TET2, or ASXL1 genes.

45. The method of claim 37, wherein the subject is determined to have CH if the subject has a variant allele frequency of ≥about 2 percent of an acquired mutation of the TET2 gene.

46. The method of claim 36, further comprising performing an assay to determine if the subject is infected with a SARS-CoV-2 virus, wherein the assay is performed prior to administering the anti-COVID-19 antibody therapy to the subject.

47. The method of claim 36, wherein the assay comprises performing viral culture to detect the SARS-CoV-2 virus.

48. The method of claim 36, wherein the assay comprises detecting nucleic acids of the SARS-CoV-2 virus.

49. The method of claim 48, wherein the assay comprises performing PCR.

50. The method of claim 36, wherein the anti-COVID-19 antibody therapy is administered to the subject in the absence of, or prior to the onset of, symptoms of acute respiratory distress syndrome (ARDS) or cytokine release syndrome (CRS).

51. The method of claim 36, wherein the anti-COVID-19 antibody therapy comprises one of more neutralizing antibodies against the spike protein of the SARS-CoV-2 virus.

52. A method of determining if a subject infected with a SARS-CoV-2 virus is a candidate for initiation of anti-COVID-19 antibody therapy, the method comprising: determining if the subject has CH, wherein, if the subject has CH the subject is a candidate for initiation of anti-COVID-19 antibody therapy.

53. The method of claim 52, wherein the step of determining if the subject has CH is performed in the absence of, or prior to the onset of, symptoms of ARDS or CRS in the subject.

54. The method of claim 52, wherein the subject is determined to have CH if the subject has a variant allele frequency of ≥about 2 percent of an acquired mutation of a leukemia-associated gene.

55. The method of claim 52, wherein the subject is determined to have CH if the subject has a variant allele frequency of ≥about 2 percent of an acquired mutation of a leukemia-associated gene in circulating leukocytes or cell-free DNA from the subject.

56. The method of claim 52, wherein the subject is determined to have CH if the subject has a variant allele frequency of ≥about 2 percent of an acquired mutation of the DNMT3A, TET2, or ASXL1 genes.

57. The method of claim 52, wherein the subject is determined to have CH if the subject has a variant allele frequency of ≥about 2 percent of an acquired mutation of the DNMT3A, TET2, or ASXL1 genes in circulating leukocytes or cell-free DNA from the subject.

58. The method of claim 52, wherein the is determined to have CH if the subject has a variant allele frequency of ≥about 2 percent of an acquired mutation of the TET2 gene.

59. The method of claim 52, wherein the is determined to have CH if the subject has a variant allele frequency of ≥about 2 percent of an acquired mutation of the TET2 gene in circulating leukocytes or cell-free DNA from the subject.

60. The method of claim 52, comprising performing an assay to determine if the subject has CH.

61. The method of claim 60, wherein the assay comprises performing a molecular analysis.

62. The method of claim 60, wherein the assay comprises performing DNA sequencing.

63. The method of claim 60, wherein the assay comprises performing DNA sequencing of DNA of circulating leukocytes from the subject or cell-free DNA from the subject.

64. The method of claim 60, wherein the assay comprises determining the variant allele frequency of an acquired mutation in a leukemia-associated gene.

65. The method of claim 60, wherein the assay comprises determining the variant allele frequency of an acquired mutation in a leukemia-associated gene in circulating leukocytes or cell-free DNA from the subject.

66. The method of claim 60, wherein the assay comprises determining the variant allele frequency of an acquired mutation in the NMT3A, TET2, or ASXL1 gene.

67. The method of claim 60, wherein the assay comprises determining the variant allele frequency of an acquired mutation in the NMT3A, TET2, or ASXL1 gene in circulating leukocytes or cell-free DNA from the subject.

68. The method of claim 60, wherein the assay comprises determining the variant allele frequency of an acquired mutation in the TET2 gene.

69. The method of claim 60, wherein the assay comprises determining the variant allele frequency of an acquired mutation in the TET2 gene in circulating leukocytes or cell-free DNA from the subject.
